(19) United States
(12) Patent Application Publication
(10) Pub. No.: US 2011/0098596 A1
(43) Pub. Date: Apr. 28, 2011

(54) Practical and Safe Needle Biopsy Device

(76) Inventors: Ali Ozkan Ozturk, Samsun (TR); Orhan Eren, Samsun (TR)

(21) Appl. No.: 12/988,509

(22) PCT Filed: Apr. 17, 2009
(86) PCT No.: PCT/TR09/00051

§ 371 Date: Dec. 29, 2010

(30) Foreign Application Priority Data
Apr. 18, 2008 (TR) 2008/02738

(51) Int. Cl. A61B 10/02

(52) U.S. Cl. 600/566; 600/567

(57) ABSTRACT

The invention relates to incision biopsy of the tissue that is collected in the tissue aspiration reservoir (19) that is administered by forward motion of the cutting inner edge (21), after the aspiration of the fluid material into the inner needle inlet (22) and the conveying space (23), and from there into the outlet (27) and the hollow of injection syringe (17) as a result of the vacuum that is created by opening of the notch (13) at the tip of the outer needle (10), with backward motion of the cutting inner needle (20) that is connected with the moveable piston that is positioned in the hollow (11) of the outer needle (10) that is connected with the said injection syringe body (16).

### TECHNICAL FIELD

The invention relates to biopsy needles which enable to take soft pieces of tissue and/or cystic fluids, tissue aspirates, and blood materials from the organs in the human body for determining the reasons of anomalous nodules or masses (tumors) in the human body, and act as a medical invasive test procedure.

Through a method that is safe, simple, and far more easier to use, as if simply administering injection, the said invention can provide possibility of administering closed biopsy to intraabdominal organs/masses that are appropriate for closed biopsy or all kinds of tissues that contain such lesions as LAP; particularly thyroid, breast, lymph node, cystic lesion, and cutaneous/subcutaneous lesions; to every medical doctor who has injection experience.

When internal organ biopsy is being administered, having the patient hold his/her breath and continue this retention throughout the duration of biopsy is very important for prevention of complications. The invention relates to a biopsy needle device that minimize complication risk due to its following properties: aim is taken once with the biopsy needle; the biopsy procedure does not include sequential manoeuvres in tru-cut biopsy, which take longer time, are consecutive, and result in longer procedures; it shortens the duration of procedure due to its being very simple; and it enables to realize whether or not the needle is in intravenous position.

### BACKGROUND OF THE INVENTION

There are two types of biopsies: open and closed. Open biopsy: Cutaneous and subcutaneous tissues are opened with a surgical procedure and the biopsy is administered to the relevant organ by a surgical method in which there is visual clue. Due to its requirement of surgical procedure, it is not useful and practical, and it is a highly invasive procedure.

Closed biopsy: It is a method which is administered with the help of needle, directed at taking pieces of tissue from target organs, less invasive, and compared to open biopsies it is used much more often in daily applications.

Needle Aspiration Biopsy: Except bone marrow biopsy which is administered with a thick needle, aspiration biopsies that are administered with a thin needle are among closed biopsies that are used much more often. Thin Needle Aspiration Biopsy (TNAB): It is a method that is administered most frequently to thyroid and lymph nodes, and one of the methods used most frequently. In this method, target lesion in target tissues such as thyroid or breast is reached with the needle and the injection syringe piston is pulled back, thus vacuum is produced inside cell or tissue aspirate containing fluid in the target tissue, and it is vacuumed first into the needle and then into the hollow of the injection syringe. The needle used here is one-piece. Cases where sufficient piece of tissue cannot be taken with the TNAB method and faulty negative results are substantially frequent.

As a Closed Biopsy Method, tru-cut biopsy is used as the second most common method, and it is frequently preferred in breast and renal biopsies. There are gun-shaped types of tru-cut which take biopsy automatically.

Other Biopsy Methods: There are also other biopsy methods such as Seldinger and Abraham, which are respectively directed at liver and pleura biopsies; these methods are not mentioned here.

Insufficiencies of the present tru-cut application method are as follows: their uses require experience and mastery. In this needle biopsy system with an inner needle and a cylindrical cannula with cutting edge which cover the inner needle;
It can be moved forward toward the target tissue when aspiration inner needle with a cavity at the tip is aligned with the tip of outer cylindrical cutter. For this reason, in order to make an incision, first the inner needle and then the outer needle must be once more made to manoeuvre within the organ.
When the needle is inserted into the organ, it is necessary to aim not at the target tissue but at a nearer location. This distance between the points that are aimed, which is nearer than the target tissue, and the target point is the same length as the length of inner needle aspiration reservoir (a few mm.). Even achieving this distance requires estimation and mastery, and it also includes margin of error of not reaching the target tissue.
When the inner needle is pushed inside, one must aim once more so that the target tissue can be reached.
The biopsy is taken by carrying out an incision procedure where outer needle cylindrical cutting tube is pushed.
Finally, the needle is pulled out. This present tru-cut biopsy method also requires a certain education and mastery.

There are many patent applications pertaining to biopsy needle devices. Among these applications, there is a patent application with number EP1903945A1. In this application, the aspiration inner tip goes out during the biopsy. It differs from the tru-cut in that the inner aspiration needle goes out and then it is retracted; that is, it is like a modified tru-cut system and the biopsy material is collected in the cavity within the inner needle. In the injection biopsy, which is our invention, there is a cutting inner needle, and there are no back and forth displacement manoeuvres in the incision procedure that is done when in the target tissue. The EP1903945A1 is not in form of an injection syringe and it does not include combination of aspiration.

On the other hand, in the patent application JP2007054449A, outer lumen is rotated with a rope style material connected to the needle, and material is taken with the tip of the needle; it seems to be a rudimentary and ineffective method. Also, it is a semi-active system that is very different from our invention.

On the other hand, in the patent application with number WO2007110812A2, there is a system which carries out coaxial incision with the help of vacuum. Aspiration notch on the inner lumen go out of the system and carry out vacuuming action, and incision is made with cutting cylinder during its re-entry. Incision method and design are different, and it does not include combination of aspiration.

### DESCRIPTION OF THE INVENTION

Most important purpose of the invention is taking of the incision biopsy by enabling incision not with outer cannula but with inner cutting needle, through a simple injection manoeuvre, by connecting inner and outer needles in the present tru-cut biopsy system with body and piston of the standard injection syringes. It includes devices, where the inner cutting needle may have one-way back and forth movements in directions of a-b as well as devices which have movements in directions of left-right or spiral movements, as variations. As an addition to this purpose of incision biopsy, another purpose is development of an injection-incision biopsy device that is practical and that can be safely applied to all soft tissue biopsies by allowing aspiration of fluid inside the mass, in case there are any, by establishing, through conveyor channel and outlet, a relationship between inlet on the inner needle and hollow in the injection syringe body.

Advantage of the invention is that it provides a simple and easy-to-use new biopsy method, which is safer and administered as if it is a regular medicine injection, without necessity of receiving a special education for injection biopsy method, as distinct from negative situations in the above mentioned tru-cut biopsy method.

Another advantage of the invention is that it enables aiming once at the target tissue and taking sufficient amount of tissue piece from the target tissue.

Another purpose of the invention is to allow its use for cystic lesions, because, due to existence of hollow channel at one edge of the inner needle, it can be used in aspiration of the fluid inside the cyst if the target tissue is cystic, that is, if it is a fluid-containing structure. With this property, it provides an advantage in taking of biopsy from cystic nodules that are frequently observed, particularly, in goiter patients.

Another purpose of the invention is that due to its being single incision biopsy that also allow thin needle aspiration system, it provides opportunity to know that the needle is in intravenous position, which is an unwanted complication of the target tissue that is reached during the biopsy procedure. In such a situation where the needle is in intravenous position, such a possibility of complication that increase the risk of haemorrhage will be known before making an incision, because blood will enter into the syringe during the action of pulling back the piston, which is the move that takes place before the incision manoeuvre, and the risk of haemorrhage, which is the most feared aspect of closed biopsy method, will be reduced.

From the viewpoint of reducing the risk of haemorrhage, another advantage of the invention is, due to entry of arterial blood into the injection syringe with pressure, in a flushing manner, particularly when the needle is in intravenous position prior to the biopsy, it is known that the needle is in intravenous position before making the incision; and reduction of the risk of heavy-fatal arterial haemorrhage, which is most feared complication in biopsies.

Another purpose of the invention is reduction of reports of insufficient material in pathological examination reports, due to the invention's being a combined higher technique that can be also used in taking incision biopsy together with the TNAB method, when it is used instead of the TNAB, particularly in such lesions as thyroid and lymph nodes, nodular, etc. where the biopsy is administered with the TNAB.

Following method will be used in order to fulfil purposes and advantages explained above: positioning of the cutting inner needle that is connected to the piston of the injection syringe in hollow of the outer needle that is connected to the body of the said injection syringe and carrying out the incision procedure of the target tissue with the cutting inner needle; incision of the tissue with tip of the cutting inner needle and taking it into the aspiration reservoir that is between the outer surface of the outer needle and the inner cutting edge, by means of opening of the notch at the tip of the outer needle of the cutting inner needle and occurring of both passive filling and vacuum effect with the opening of the notch through retraction of the said moveable cutting inner needle by means of the outer needle that is inserted into the target tissue in the human body.

In addition to the incision biopsy explained above, in order to fulfil the purpose of aspiration, it contains a connection head that has a cavity that is formed on the cutting inner needle and an inlet that is formed at the cutting edge of the cutting inner needle; for transfer of tissue aspirate, cystic fluid, and blood elements that are first taken into conveying space; into the injection syringe hollow of the injection syringe body by means of the cutting inner needle.

### BRIEF DESCRIPTION OF THE FIGURES

In order to fully understand the construct of the present invention and its advantages due to additional elements, it must be evaluated with the figures that are explained below.

FIG. 1; Complete two-dimensional side assembly view of the biopsy needle device that is the subject of the invention, when the inner needle positioned in the outer needle is retracted in direction b.

FIG. 2; Two-dimensional side view of the outer needle of the biopsy needle device that is the subject of the invention.

FIG. 3; Two-dimensional side view of the inner needle of the biopsy needle device that is the subject of the invention.

FIG. 4; Complete two-dimensional side assembly view of the biopsy needle device that is the subject of the invention, when the inner needle positioned in the outer needle is moved forward in direction a.

FIG. 5; Two-dimensional disassembly view of the outer needle when it is separated from the body of the injection syringe.

FIG. 6; Two-dimensional disassembly view of the inner needle when it is separated from the moveable body of the injection syringe.

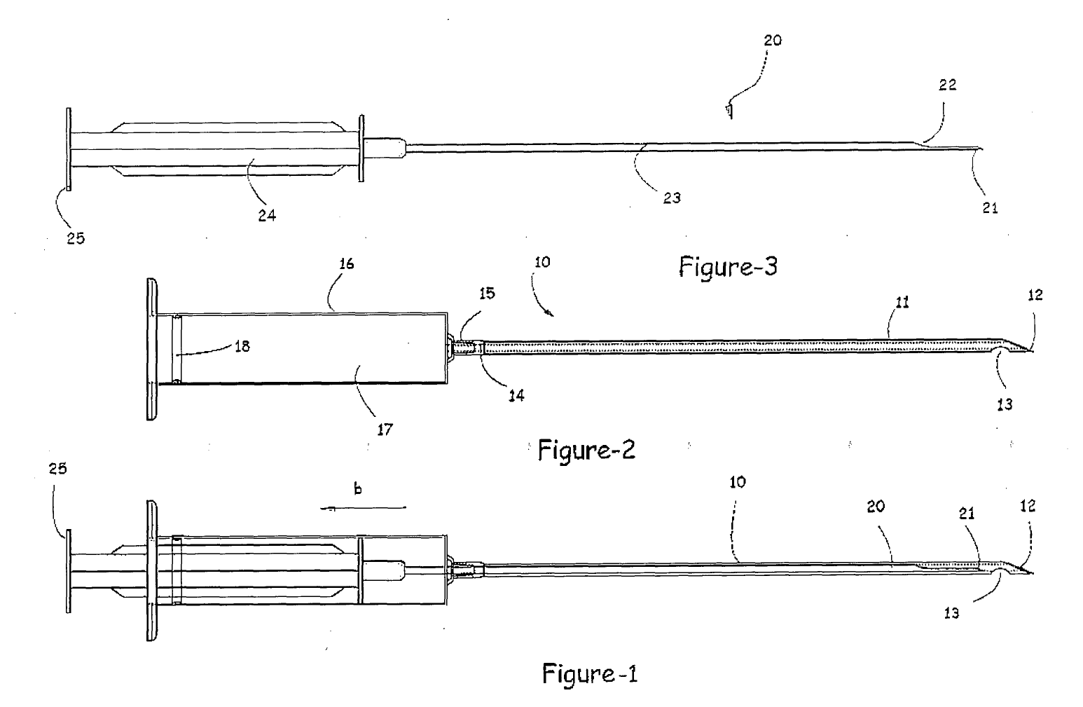

### REFERENCE NUMBERS

10-Outer needle (cannula)

11-Hollow

12-Needle tip

13-Notch

14-Needle connection head

15-Head connection surface

16-Injection syringe body

17-Hollow of the injection syringe

18-Impermeability element

19-Tissue aspiration reservoir

20-Cutting inner needle

21-Cutting edge

22-Inlet

23-Conveying space

23.1-Transition channel

24-Moveable piston

25-Pressure surface

26-Connection head

27-Outlet

### DETAILED DESCRIPTION OF THE INVENTION

In respect of its structure pertaining to the present art, the invention contains an injection syringe body (16), injection syringe hollow (17) that is formed inside the said body (16) and impermeability element (18) that is positioned in this hollow (17), moveable piston (24) and pressure surface (25) that is formed on this piston (24).

In respect of its innovation property, the said invention includes steps which enable taking of incision biopsy and/or tissue aspirate, cystic fluid, and blood material from anomalous nodules or masses (tumors) in the human body by means of the biopsy needle device.

In order to ensure aiming in one go and easy use of the biopsy needle device, these steps include injection incision procedure which consists of the following steps: positioning of the cutting inner needle (20) in hollow (11) of the outer needle (10) that is connected to the body (16) of the said injection syringe and carrying out the incision procedure of the target tissue with the cutting inner needle (20); opening of the notch (13) at the tip of the outer needle (10) with the cutting inner needle (20) through retraction of the said moveable body (24) with its movement in direction b (FIG. 6) by means of the outer needle (10) that is inserted into the target tissue in the human body and occurrence of both passive filling and vacuum effect with the opening of the notch (13) and taking of the tissue into the tissue aspiration reservoir (19) by means of the cutting edge (21) inlet.

In addition to the incision biopsy explained above, in order to carry out the aspiration procedure, it contains a connection head (26) that has an outlet (27) that is formed on the cutting inner needle (20) and an inlet (22) that is formed at the cutting edge (21) of the cutting inner needle (20); for transfer, through conveying space (23), of such samples as cystic fluid, tissue aspirate, and blood elements taken with the vacuum effect that is created by opening of the cutting inner needle (20) into the injection syringe hollow (17) of the said injection syringe body (16). For making this aspiration procedure easier, the system can be integrated with valve system that enables unidirectional fluid passage.

Usage and manner of working of the device is as follows:

In this device, the most important component and the most important difference compared to the existing devices is that the procedure of incision and taking in of the tissue is made by the cutting needle (20), and the cutting needle (20) which assumes this role is positioned inside the outer needle (10). On the other hand, another important aspect is that the outer needle (10) takes on the role of insertion into the patient's body and after being locked into the target it carries out bedding function for rectilinear motion of the cutting needle (20) (in directions a-b) by remaining in a fixed position inside the hollow (11), and it provides the tissue aspiration reservoir (19) for the notch (13) that it contains at its terminal side and the target piece of tissue.

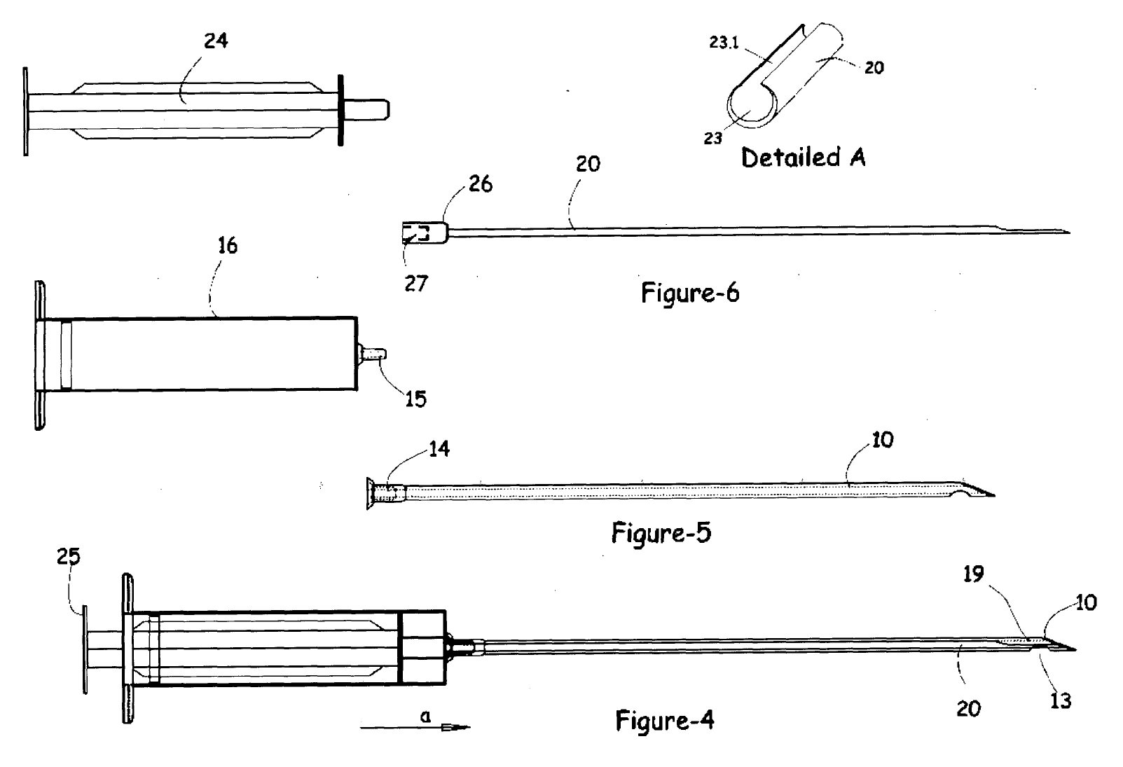

In respect of its position in FIG. 4, the biopsy needle device is inserted into the patient's body in closed state. In closed position, the cutting needle (20) closes aperture space of the notch (13). When the moveable. body (24) is retracted (in direction b) by means of the pressure surface (25), it begins to open the aperture space of the notch (13). (See FIG. 1) When the retraction process is continuing, due to vacuuming, the piece of tissue is first directed into the conveying space (23) of the cutting needle (20); and thus, filling of such materials as cystic fluid, blood, and tissue aspirate, in case there are any, into the hollow of the injection syringe (17) is achieved. Following this, the moveable piston (24) is pushed forward, the piece of tissue that enter into the notch (13) is incised and taken into the tissue aspiration reservoir (19), and when the outer needle notch is in closed position, where is it closed by the inner needle as shown in FIG. 4, the needle is completely pulled out of the body. The said notch (13) may have different geometrical forms and sizes. It may contain more than one notch (13). Needle tip (12) of the outer needle (10) may be open or closed.

As mentioned above, the piece of tissue that is taken from the patient is first directed toward the tissue aspiration reservoir (19) of the cutting needle (20). If there are liquid/semi-liquid fluids such as cystic fluids, tissue aspirates, and blood elements here, these are made to fill into the conveying space (23) by means of the inlet (22) that is formed at the terminal side of the cutting needle (20), and from there into the hollow of injection syringe (17) through the outlet (19) and the connection head (26). The conveying space (23) also has a transition channel (23.1). Preferably, it has the appearance of a crescent, and preferably it can be produced in this shape. (See detail a)

Area of protection of this invention is determined in the claims section and it cannot be limited to what is certainly explained with purpose of providing an example. It is clear that an. expert can produce the innovation in the art also by using similar constructs and/or an expert can apply this construct to other areas, with similar purposes, used in the related art. Therefore, it is also obvious that such constructs will lack the criteria of innovation and particularly transgression of the present situation of the art.

1.-4. (canceled)

5. A needle biopsy device comprising an injection syringe body (16), hollow (17) of the injection syringe which is formed inside the said body (16) and impermeability element (18) which is positioned in this hollow (17), moveable piston (24), pressure surface (25) which is formed on this piston (24), outer needle (10) which is connected to the injection syringe body (16), hollow (11) which is located in the outer needle (10) and characterized by the inner needle (20) which is positioned in the hollow (11) of the outer needle (10) and which enables to cut and take in the tissue, needle tip (12), notch (13) which is located at the tip of the inner needle (20), needle connection head (14) which interconnects the injection syringe body (16) and the needle (10) by means of head connection surface (15), tissue application reservoir (19) which is located between the cutting inner needle (20) and the hollow (11), cutting edge (21) which forms the tip of the cutting inner needle (20), inlet (22), conveying space (23) wherein the samples are transferred, connection head (26) and outlet (27).

6. A needle biopsy device according to claim 1 characterized by a notch (13) which is located at the lower end of the outer needle (10) and opened by the motion of the cutting inner needle (20) and wherein vacuum is formed by pulling on the cutting edge (21) and target tissue piece is collected in thereof.

7. A needle biopsy device according to claims 1 to 2, characterized by a tissue application reservoir (19) which is located between the outer needle (10) and the cutting inner needle (20) and in which target tissue piece is collected.

8. A needle biopsy device according to claims 1 to 3, characterized by a cutting edge (21) which enables the target tissue settling on the notch (13) to be cut.

9. A needle biopsy device according to claims 1 to 4, characterized by inlet (22) which enables cystic fluid, tissue aspirate, blood elements, fluid/semi-fluids to be filled firstly to the conveying space (23) then to the hollow of the injector syringe (17) over the outlet (27) and the connection head (26) from there.

10. A needle biopsy device according to claims 1 to 5, characterized by needle tip (12) which can be open or closed.

11. A needle biopsy device according to claims 1 to 6, characterized by a plurality of notches (13) which can have different forms and sizes.
